SEQUENCE LISTING (1 sequence)

SEQ ID NO: 1 - DNA, 10 bp, Artificial Sequence
  Description of Artificial Sequence Synthetic primer
aaaaaaaaaa

(19) United States
(12) Patent Application Publication
(10) Pub. No.: US 2013/0324419 A1
(43) Pub. Date: Dec. 5, 2013

(54) METHODS FOR NUCLEIC ACID CAPTURE AND SEQUENCING

(71) Applicant: Somasekar Seshagiri, San Carlos, CA (US)

(72) Inventor: Somasekar Seshagiri, San Carlos, CA (US)

(21) Appl. No.: 13/819,657

(22) PCT Filed: Aug. 25, 2011
(86) PCT No.: PCT/US2011/049151

§ 371 Date: Aug. 15, 2013

Related U.S. Application Data
(60) Provisional application No. 61/402,350, filed on Aug. 27, 2010.

(51) Int. Cl. C12Q 1/68; C12N 15/10

(52) U.S. Cl. CPC C12Q 1/6874; C12N 15/1072
USPC 506/2; 435/6.1; 435/6.11

(57) ABSTRACT

Methods of capturing and sequencing target nucleic acid molecules are provided. Methods of determining the methylation status of genomic DNA are also provided.

### RELATED APPLICATIONS

This application claims the benefit under 35 USC 119(e) of U.S. Provisional Application No. 61/402,350 filed Aug. 27, 2010, the contents of which are incorporated herein by reference.

### FIELD OF THE INVENTION

The present invention relates to separating target nucleic acid molecules from nucleic acid mixtures and determining the sequence of such molecules.

### BACKGROUND

Since the completion of the entire human genome sequence, genomics research has shifted toward “resequencing” efforts in which variations, e.g., disease-associated mutations, are identified within and across genomes. Resequencing of “partitioned” genomes enriched for particular regions of interest, e.g., exons, has required several steps involving the “capture” and sequencing of those regions. For example, in certain microarray-based methods, genomic DNA is sheared into fragments of a particular size range; the fragments are end-repaired, ligated to unique adaptors, and amplified; the amplified fragments are then captured using a microarray containing probes complementary to reference genomic sequence of interest; the captured (hybridized) fragments are eluted and amplified; and the amplified fragments are sequenced, e.g., using “next generation” sequencing technologies or resequencing arrays. See, e.g., WO 2008/115185; Okou et al. (2007) Nature Methods 4:907-909; Hodges et al. (2007) Nature Genetics 39:1522-1527. A reduction in the steps required for separating and sequencing nucleic acids of interest would increase efficiency and accuracy and potentially lower costs. The present invention meets this need and provides additional benefits.

Cytosine methylation, generally occurring at CpG dinucleotides in the genome, plays an important role in gene regulation and epigenetic inheritance. Certain existing methods for determining the methylation state of a genomic region utilize bisulfite treatment. In such methods, exposure of denatured genomic DNA to bisulfite ion results in the deamination of cytosine to uracil, whereas methylated cytosines are protected from this conversion. The absence or presence of a conversion event may be detected, e.g., by next generation sequencing methods or by using probe arrays. See, e.g., WO 2010/085343. Such methods often entail several steps, e.g., amplification, capture, elution and sequencing of bisulfite-treated DNA, or they alternatively require designing probes to interrogate the absence or presence of a conversion event at every genomic region of interest, which may be costly and labor intensive. Moreover, such methods do not assess methylation state at the level of individual DNA molecules but instead look at populations of nucleic acid molecules corresponding to a particular genomic region of interest. The present invention provides a more efficient and accurate method of assessing methylation status of genomic DNA, thus satisfying a need in the art and providing other benefits.

### SUMMARY

In one aspect, a method of capturing and sequencing a target nucleic acid molecule is provided, the method comprising (a) exposing a solid support to a mixture of nucleic acids comprising the target nucleic acid molecule under hybridizing conditions, wherein the target nucleic acid molecule forms a specific hybridization complex with a primer immobilized on the solid support in a priming-competent configuration; (b) separating unbound and non-specifically bound nucleic acids from the solid support; (c) exposing the solid support to a polymerase and nucleotides under polymerization conditions; and (d) determining a nucleic acid sequence of the target nucleic acid molecule by detecting nucleic acid polymerization from the immobilized primer by the polymerase using the target nucleic acid molecule as a template.

In one embodiment, the target nucleic acid molecule is from a region of genomic DNA. In one such embodiment, the target nucleic acid comprises all or part of an exon. In another embodiment, the target nucleic acid molecule is RNA, the polymerase is a reverse transcriptase, and the primer comprises a 3′ poly-T sequence. In another embodiment, the target nucleic acid molecule is DNA, and the polymerase is a DNA polymerase. In another embodiment, the nucleotides are labeled at their terminal phosphates. In one such embodiment, the polymerase is labeled with a FRET donor, and the nucleotides are labeled with a FRET acceptor. In one such embodiment, the FRET donor is a fluorescent nanoparticle.

In a further aspect, a method of determining the methylation status of a genomic DNA fragment is provided, the method comprising (a) immobilizing a genomic DNA fragment on a solid support; (b) determining a nucleic acid sequence of the immobilized genomic DNA fragment on the solid support by detecting nucleic acid polymerization by a polymerase using the immobilized genomic DNA fragment as a template; (c) subjecting the immobilized genomic DNA fragment to bisulfite treatment; (d) determining a nucleic acid sequence of the immobilized, bisulfite-treated genomic DNA fragment on the solid support by detecting nucleic acid polymerization by a polymerase using the immobilized, bisulfite-treated genomic DNA fragment as a template; (e) comparing the nucleic acid sequence determined in (b) with the sequence determined in (d), wherein conversion of a cytosine residue in the genomic DNA fragment indicates that the residue was unmethylated in the genomic DNA fragment prior to the bisulfite treatment, and wherein absence of conversion of a cytosine residue in the genomic DNA fragment indicates that the residue was methylated in the genomic DNA fragment prior to the bisulfite treatment.

In one embodiment, the genomic DNA fragment is immobilized to the solid support by an adaptor. In one such embodiment, the adaptor comprises a primer binding site, and cytosines in the primer binding site are protected. In one such embodiment, the polymerase of (b) and/or (d) polymerizes a nucleic acid strand from a primer annealed to the primer binding site. In another embodiment, nucleic acid polymerization in (b) and/or (d) is detected by detecting the incorporation of labeled nucleotides. In one such embodiment, the labeled nucleotides are labeled at their terminal phosphates. In one such embodiment, the polymerase of (b) and/or (d) is labeled with a FRET donor, and the nucleotides are labeled with a FRET acceptor. In one such embodiment, the FRET donor is a fluorescent nanoparticle.

### BRIEF DESCRIPTION OF THE FIGURES

FIG. 1 depicts the direct capture of select regions of sheared DNA on an oligonucleotide array using complementary oligonucleotides and direct sequencing of the captured DNA by using the free 3′ end of the oligonucleotide used to capture the DNA, a labeled polymerase and labeled dNTPs that will allow direct monitoring of the bases (sequence) added and the polymerase during real-time synthesis of DNA.

FIG. 2 depicts the direct capture of select regions of sheared DNA using beads coated with complementary oligonucleotides followed by arraying the beads and sequencing of the captured DNA by using the free 3′ end of the oligonucoleotide used to capture the DNA, a labeled polymerase and labeled dNTPs that will allow direct monitoring of the bases (sequence) added and the polymerase during real-time synthesis of DNA.

FIG. 3 depicts the direct capture of RNA on a poly-dT oligonucleotide array and sequencing of the captured RNA by converting it into cDNA using the free 3′ end of the poly-dT oligonucleotide and reverse transcriptase. After cDNA conversion the cDNA is sequenced using a poly-A primer, a labeled polymerase and labeled dNTPs that will allow direct monitoring of the bases (sequence) added and the polymerase during real-time synthesis of DNA. Alternatively, the captured RNA can be sequenced directly to obtain the sequence using a labeled reverse transcriptase and labeled dNTPs that will allow direct monitoring of the bases (sequence) added and the reverse transcriptase during real-time synthesis of DNA.

FIG. 4 depicts the direct capture of RNA using poly-dT oligonucleotides immobilized on beads, arraying the beads on a surface and then sequencing the captured RNA by converting into cDNA using the free 3′ end of the poly-dT oligonucleotide and reverse transcriptase. After cDNA conversion, the cDNA is sequenced using a poly-A primer, a labeled polymerase and labeled dNTPs that allow direct monitoring of the bases (sequence) added and the polymerase during real-time synthesis of DNA. Alternatively, the captured RNA can be sequenced directly to obtain the sequence using a labeled reverse transcriptase and labeled dNTPs that allow direct monitoring of the bases (sequence) added and the reverse transcriptase during real-time synthesis of DNA.

FIG. 5 depicts nucleic acid methylation status determination by sequencing the same DNA molecule on an array in successive reactions where the second round of sequencing is done after bisulfite treatment to allow conversion of methylated cytosines to uracil. An appropriate primer, a labeled polymerase and labeled dNTPs allow direct monitoring of the bases (sequence) added and the polymerase during real-time synthesis of DNA.

FIG. 6 depicts nucleic acid methylation status determination by sequencing the same DNA molecule on a bead in successive reactions where the second round of sequencing is done after bisulfite treatment to allow conversion of methylated cytosines to uracil. An appropriate primer, a labeled polymerase and labeled dNTPs allow direct monitoring of the bases (sequence) added and the polymerase during real-time synthesis of DNA.

### DETAILED DESCRIPTION OF EMBODIMENTS OF THE INVENTION

### I. Definitions

“Bisulfite treatment” refers to exposure of a nucleic acid to bisulfite ion (e.g., magnesium bisulfite or sodium bisulfite) at a concentration sufficient to convert unprotected cytosines to uracils. “Bisulfite treatment” also refers to exposure of a nucleic acid to other reagents that can be used to convert unprotected cytosines to uracils, e.g., disulfite and hydrogensulfite, at an appropriate concentration. “Bisulfite treatment” generally includes exposure of the nucleic acid to a base, e.g., NaOH, after exposure to the bisulfite ion or other reagent.

“Conversion of a cytosine residue” refers to the conversion of a cytosine residue to a uracil residue as a result of bisulfite treatement.

“Exon” refers to a coding region of a genome.

“Hybridizing conditions” refers to conditions permissive for hybridization of complementary nucleic acid strands.

“Determining a nucleic acid sequence” refers to determining the identity of at least one nucleotide, and in some embodiments a plurality of nucleotides, of a target nucleic acid molecule.

“Immobilized” and “immobilizing” refers to the attachment of a nucleic acid to a solid support, either directly or indirectly, by means other than complementary base pairing. A specific hybridization complex is considered immobilized to a solid support if at least one of two nucleic acid strands in a specific hybridization complex is “immobilized” to the solid support as defined above.

“Label” refers to any moiety that can be detected directly or indirectly.

“Nucleic acid” refers to polymers of nucleotides of any length.

“Nucleotide” refers to nucleotides and analogs thereof that are capable of being incorporated into a growing nucleic acid strand by a polymerase. Nucleotides include but are not limited to the four types of nucleotides generally incorporated into DNA (adenine, guanine, cytosine, and thymine); the four types of nucleotides generally incorporated into RNA (adenine, guanine, cytosine, and uracil); nucleotides with modified bases such as inosine; and nucleotides that are labeled or otherwise modified.

“Polymerase” refers to an enzyme, whether naturally or non-naturally occurring, or an enzymatically active fragment thereof, that is capable of incorporating nucleotides into a growing nucleic acid strand under polymerization conditions, including but not limited to DNA polymerases, RNA polymerases, and reverse transcriptases.

“Polymerization conditions” refers to conditions permissive for a polymerase to incorporate nucleotides into a growing nucleic acid strand.

“Primer” refers to a nucleic acid to which nucleotides may be added by a polymerase. “Added” refers to addition of a nucleotide directly to the primer by the polymerase as well as subsequent addition of nucleotides to the growing nucleic acid strand originating from the primer.

“Priming-competent configuration” refers to a primer having an available reactive group to which a polymerase can add a nucleotide.

“Solid support” refers to any solid substrate.

“Specific hybridization complex” refers to a hybridization complex capable of forming or being substantially maintained under stringent hybridization conditions and/or stringent wash conditions.

“Target nucleic acid molecule” refers to any nucleic acid molecule of interest.

“Template” refers to a single-stranded nucleic acid, or a denatured region of a double-stranded nucleic acid, that a polymerase can utilize to synthesize a complementary nucleic acid strand.

### II. Capture and Sequencing of Target Nucleic Acid Molecules

In one aspect, the present invention relates to a method of capturing a target nucleic acid molecule using a complementary nucleic acid, e.g. a primer, immobilized on a solid support. In certain embodiments, the nucleic acid sequence of the target nucleic acid molecule is then determined by detecting nucleic acid polymerization from the primer by a polymerase that uses the target nucleic acid molecule as a template. The detection occurs at the single-molecule level, and in real time or near-real time.

### Target Nucleic Acid Molecules

In various embodiments, the target nucleic acid molecule is DNA. In one embodiment, the target nucleic acid molecule may correspond to any region of a genome (the “target region”), such as a human genome or a genome from any other organism. The target region may be one or more continuous blocks of several megabases, or several smaller contiguous or discontiguous regions such as all of the exons from one or more chromosomes, or sites known to contain SNPs. The genome containing the target region may be partial or complete. The genome may be derived from any biological source, such as a patient sample or pooled patient sample; cell lines or cell cultures; biopsy material; normal tissue samples or samples from tumors or other diseased tissue; and other biological sources that would be appreciated by one skilled in the art. In one embodiment, genomic DNA containing the target nucleic acid is sheared, e.g., by sonication or hydrodynamic force, into fragments, generally of about 200-600 base pairs, and the target nucleic acid molecule is captured from the fragments or a fractionated portion thereof. In another embodiment, the target nucleic acid molecule may be coding or non-coding sequence. In one such embodiment, the target nucleic acid molecule is an exon or portion thereof.

In various embodiments, the target nucleic acid molecule is RNA. In one embodiment, the target nucleic acid is an mRNA transcript or portion thereof. In one such embodiment, the target nucleic acid is an mRNA transcript or portion thereof having a poly-A tail. The presence of a poly-A tail may allow for hybridization to a probe or primer comprising a poly-T sequence of sufficient length, generally at the 3′ end of a primer. In a further embodiment, the target nucleic molecule is cDNA generated from mRNA, e.g., by reverse transcriptase.

### Capture

In various embodiments, the target nucleic acid molecule is captured from a mixture of nucleic acid, e.g., RNA, DNA (e.g., genomic DNA), or cDNA molecules. In one embodiment, the nucleic acids in the mixture are amplified prior to capture of the target nucleic acid molecule. This may be achieved, e.g., by ligating adaptors containing universal priming sites to the termini of the nucleic acid molecules in the mixture, where the termini may optionally undergo end-repair prior to the ligation. Universal primers can thus be used to amplify the nucleic acids in the mixture.

In various embodiments, the target nucleic acid molecule is captured using a complementary nucleic acid immobilized on a solid support. The complementary nucleic acid need not be completely complementary to the target nucleic acid molecule, but may contain mismatches, so long as the target nucleic acid molecule and the complementary nucleic acid molecule are capable of forming a specific hybridization complex. In one embodiment, the complementary nucleic acid is a primer. In one such embodiment, the primer is immobilized on the solid support in a priming-competent configuration. For example, a primer having a 3′-OH is immobilized on the solid support wherein the 3′-OH is available to a polymerase for addition of nucleotides to the 3′ end of the primer. This may be achieved, e.g., by immobilizing the primer to the solid support by its 5′ end or by an internal region of the primer, so long as the primer is priming-competent. A primer may be of any length, so long as it is capable of forming a specific hybridization complex with a target nucleic acid molecule, and in certain embodiments, a primer is at least 10, 15, 20, 25, 50, 75, 100, 200, 300, 400, or 500 base pairs in length.

The art is familiar with methods for immobilizing nucleic acids onto solid supports. For example, nucleic acids, such as the complementary nucleic acids provided above, may be immobilized on the solid support by covalent or non-covalent linkage. Suitable chemical linkers and other linkages are known to those skilled in the art. For example, in one embodiment, the nucleic acid to be immobilized is biotinylated (e.g., contains one or more biotinylated nucleotides), and the solid support has streptavadin on its surface, wherein the biotin moiety of the nucleic acid binds to the streptavadin, thus immobilizing the nucleic acid.

In a further embodiment, immobilization as provided in the embodiments above is achieved by synthesizing the nucleic acid on the solid support. For example, a primer may be synthesized on a solid support by polymerizing nucleotides in a 5′ to 3′ direction, leaving an available 3′-OH at the primer terminus distal to the solid support. Chemical methods for synthesizing oligonucleotides in a 5′ to 3′ direction on a solid support, such as a high density microarray, are known in the art and may be utilized for the purposes described herein. See, e.g., Albert et al. (2003) “Light directed 5′→3′ synthesis of complex oligonucleotide microarrays,” Nucleic Acids Res. 31(7):e35, incorporated by reference herein in its entirety.

In various embodiments, the solid support is any substrate to which a nucleic acid may be immobilized. Such substrates include but are not limited to glass (e.g., glass microscope slides), metal, ceramic, polymeric beads, and other substrates. In certain embodiments, the solid support is in the form of an array, e.g., a microarray. In certain embodiments, a nucleic acid may be immobilized on a solid support, e.g., a bead, which in turn is captured or otherwise immobilized on another solid support, e.g., a glass slide or microarray.

In certain embodiments, a solid support on which a complementary nucleic acid is immobilized is exposed to a mixture of nucleic acids containing the target nucleic acid molecule under hybridizing conditions. The target nucleic acid molecule thus forms a specific hybridization complex with the complementary nucleic acid. In further embodiments, the solid support is washed to remove unbound and non-specifically bound nucleic acids, thereby separating the target nucleic acid molecule (contained within the specific hybridization complex) from other nucleic acids in the mixture. In certain embodiments, the exposing of the solid support to the mixture of nucleic acids and/or the washing of the solid support takes place under stringent hybridization conditions and/or stringent wash conditions, respectively.

As used herein, “hybridization” refers to the pairing of complementary nucleic acid strands. Hybridization and the strength of hybridization (i.e., the strength of the association between the nucleic acid strands) is affected by such factors as the degree of complementarity between the nucleic acids, stringency of the conditions, the Tm of the hybridization complex, and the G:C ratio of the nucleic acids. While the invention is not limited to a particular set of hybridization conditions, stringent hybridization conditions may be employed. Stringent hybridization conditions may be determined empirically by one skilled in the art using routine methods. Stringent hybridization conditions are sequence-dependent and also depend on environmental factors such as salt concentration and the presence of organic solvent. Generally, stringent hybridization conditions are selected to be about 5° C. to 20° C. lower than the thermal melting point (Tm) for a specific nucleic acid sequence at a defined ionic strength and pH. In certain embodiments, stringent hybridization conditions are about 5° C. to 10° C. lower than the thermal melting point for a specific nucleic acid bound to a complementary nucleic acid. The Tm is the temperature (under defined ionic strength and pH) at which 50% of a nucleic acid (e.g., a target nucleic acid molecule) hybridizes to a perfectly matched primer.

Similarly, stringent wash conditions may be determined empirically by one skilled in the art using routine methods. For example, stringent wash conditions may be ascertained that allow separation of non-specifically bound nucleic acids from specific hybridization complexes immobilized on a solid support, e.g., an array. In one embodiment, an array is exposed to hybridization conditions (e.g., stringent hybridization conditions) and then washed with buffers containing successively lower concentrations of salts, and/or higher concentrations of detergents, and/or at increasing temperatures until the signal-to-noise ration for specific to non-specific hybridization is high enough to facilitate detection of specific hybridization, e.g., hybridization between nucleic acid strands that share complete or substantially complete complementarity. In certain embodiments, stringent wash conditions will include temperatures of about 30° C., 37° C., 42° C., 45° C., 50° C., or 55° C. In certain embodiments, stringent wash conditions will include salt concentrations of ≦1M, ≦500 mM, ≦250 mM, ≦100 mM, ≦50 mM, or ≦25 mM, but ≧10 mM. An example of stringent hybridization conditions is as follows: 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C. An example of stringent wash conditions is as follows: 0.1×SSC containing EDTA at 55° C.

### Sequence Determination

Following separation of the target nucleic acid molecule (contained within the specific hybridization complex) from other nucleic acids in the mixture, the target nucleic acid molecule may be sequenced. In various embodiments, this step is generally referred to as “resequencing,” where the sequence of the target region from a reference genome is already known. Current methods for resequencing require elution of the target nucleic acid molecule from the specific hybridization complex followed by amplification of the target nucleic acid molecule, and the amplified target nucleic acid molecules are then sequenced using “next generation” sequencing technologies (e.g., sequencing platforms capable of parallel, high throughput sequencing) or resequencing arrays (microarrays containing probes the specifically detect the absence or presence of mutations in discrete segments of a nucleic acid). The requirement for elution and amplification steps is time- and resource-consuming, and it may lead to loss of sample or otherwise bias representation of an individual target nucleic acid molecule within a population of target nucleic acid molecules.

Thus, in various embodiments, the resequencing of a target nucleic acid molecule occurs without eluting it from the specific hybridization complex. This may be achieved, in one embodiment, where the complementary nucleic acid, which is contained within the specific hybridization complex, is a primer immobilized on the solid substrate in a priming-competent configuration. For example, the primer may hybridize to a particular region of a target nucleic acid molecule, with the remainder of the target nucleic acid molecule being in single-stranded form. Accordingly, a polymerase would be capable of adding a nucleotide to the available 3′-OH of the primer using the single-stranded (unhybridized) portion of the target nucleic acid molecule as a template. The nucleic acid strand synthesized by the polymerase is used to determine the sequence of the target nucleic acid molecule.

Accordingly, the solid support, on which the specific hybridization complex is immobilized via the primer, may be exposed to a polymerization reaction mixture. In one embodiment, the polymerization reaction mixture comprises a polymerase and nucleotides. The polymerase may be a DNA polymerase, RNA polymerase, or reverse transcriptase. Where the target nucleic acid molecule is RNA, the polymerase may be a reverse transcriptase. In other embodiments where the target nucleic acid molecule is DNA, the polymerase is DNA polymerase. Certain exemplary DNA polymerases include but are not limited to bacterial DNA polymerases (e.g., E. coli DNA pol I, II, III, IV, and V, and the Klenow fragment of DNA pol I); viral DNA polymerases (e.g., T4 and T7 DNA polymerases); archaeal DNA polymerases (e.g., Thermus aquaticus (Taq) DNA polymerase, Pyrococcus furiosus (Pfu) DNA polymerase, “Deep Vent” DNA polymerase (New England BioLabs)); eukaryotic DNA polymerases; and engineered or modified variants thereof. Certain exemplary RNA polymerases include but are not limited to T7, T3 and SP6 RNA polymerases, and engineered or modified variants thereof. Certain exemplary reverse transcriptases include but are not limited to reverse transcriptases from HIV, MMLV, and AMV, as well as commercially available reverse transcriptases such as SUPERSCRIPT (Invitrogen, Carlsbad, Calif.).

In certain embodiments, the nucleotides and/or the polymerases in a polymerization reaction mixture are labeled. In one embodiment, one, two, three, or four types of nucleotides are differentially labeled. In one such embodiment, four different types of nucleotides are labeled with four different labels. For example, in the case of dNTPs, adenine (or a functionally equivalent analog), guanine (or a functionally equivalent analog), cytosine (or a functionally equivalent analog), and thymine (or a functionally equivalent analog) are each labeled with a different label, e.g., a different fluorophore. Likewise, in the case of rNTPs, adenine (or a functionally equivalent analog), guanine (or a functionally equivalent analog), cytosine (or a functionally equivalent analog), and uracil (or a functionally equivalent analog) are each labeled with a different label, e.g., a different fluorophore. Suitable labels include but are not limited to luminescent, photoluminescent, electroluminescent, bioluminescent, chemiluminescent, fluorescent, and/or phosphorescent labels. Fluorescent labels include but are not limited to xanthine dye, fluorescein, cyanine, rhodamine, coumarin, acridine, Texas Red dye, BODIPY, ALEXA, GFP, and modifications thereof. A label may be directly attached to a nucleotide or may be attached via a suitable linker. A label may be attached to a nucleotide at any position that does not significantly interfere with the ability of a polymerase to incorporate the nucleotide into a growing nucleic acid strand. In one embodiment, the label is attached to a phosphate of the nucleotide, e.g., the terminal phosphate of a nucleotide, wherein the phosphate chain of the nucleotide, and therefore the label, is cleaved upon incorporation of the nucleotide into a growing nucleic acid strand. The labeled nucleotides and/or polymerases may allow for the detection of the nucleic acid strand synthesized by the polymerase.

In certain embodiments, the sequence of the target nucleic acid molecule is determined by identifying the nucleotides that are incorporated into a growing nucleic acid strand by a polymerase, in the order in which they are incorporated. One embodiment comprises directly or indirectly detecting the labels of the nucleotides that are incorporated into a growing nucleic acid strand, in the order in which they are incorporated, and correlating the detected labels with the identity of the nucleotides, thereby ascertaining the sequence of the growing nucleic acid strand. Thus the sequence of the target nucleic acid molecule (or the complement thereof, depending on whether the primer hybridizes to the sense or antisense strand of the target nucleic acid molecule) is determined. In such embodiments, the detection of the labels, and in principle the sequencing of the target nucleic acid molecule, takes place in real-time and at the “single molecule” level. It is noted above and further exemplified below that the label may be removed coincidentally with the incorporation of the nucleotide into the growing nucleic acid strand, such that the resulting nucleic acid strand is not labeled.

Certain methods for detecting labeled nucleotides as they are incorporated into a growing nucleic acid strand are described in WO 2010/002939. Such methods rely on Forster Resonance Energy Transfer (FRET) between a “donor” molecule (FRET donor) and an “acceptor” molecule (FRET acceptor) when the molecules are in sufficient proximity to one another. In particular embodiments, a polymerase is labeled with a FRET donor fluorophore, and a nucleotide is labeled with a FRET acceptor fluorophore. As the polymerase incorporates the nucleotide into a growing nucleic acid strand, the FRET donor and acceptor fluorophores are brought into proximity, allowing the transfer of energy from the FRET donor fluorophore to the FRET acceptor fluorophore. The energy transfer decreases the emission intensity of the FRET donor fluorophore and increases the emission intensity of the FRET acceptor fluorophore. Detection of the emission spectrum of the FRET acceptor indicates the identity of the nucleotide being incorporated. In one embodiment, the FRET donor attached to a polymerase is a fluorescent nanoparticle, e.g., a nanocrystal, and more specifically, a quantum dot, as described in WO 2010/002939. A FRET donor may be illuminated with an excitation source such as a laser wherein the donor emission is produced. In further embodiments, a different FRET acceptor is attached to each of one or more types of nucleotides, and in particular each of three or four types of nucleotides. A FRET acceptor may be any of the fluorescent labels discussed above.

Labels may be detected using any suitable method or device including but not limited to charge couple devices and total internal reflection microscopy.

In certain embodiments, where the target nucleic acid molecule is polyA-RNA, the target nucleic acid molecule is captured using a primer comprising a poly-T sequence. cDNA is synthesized (but not sequenced) from the primer using reverse transcriptase. The target nucleic acid molecule is then denatured from the specific hybridization complex on the solid support, leaving the newly synthesized cDNA strand, now immobilized on the solid support via the primer. The solid support is then exposed to a primer comprising poly-A, which hybridizes to the newly synthesized cDNA. The newly synthesized cDNA is sequenced by exposing the solid support to a polymerization reaction mixture, as provided above, wherein a DNA polymerase synthesizes a nucleic acid strand from the polyA primer.

Using the above methods, multiple target nucleic acid molecules may be separated and sequenced by selecting primers specific for each target nucleic acid molecule of interest, and immobilizing the primers on discrete areas of a solid support, e.g., on a microarray. In this manner, target nucleic acid molecules of interest may be separated and sequenced in a high throughput manner.

### III. Determination of Methylation Status

In another aspect, the present invention relates to a method of determining the methylation status of CpG dinucleotides within a genomic DNA fragment by immobilizing the genomic DNA fragment to a solid support; determining a nucleic acid sequence of the immobilized genomic DNA fragment by detecting polymerization of nucleotides by a polymerase that uses the genomic DNA fragment as a template; denaturing the immobilized genomic DNA fragment from the newly synthesized nucleic acid strand; exposing the solid support to bisulfite; and determining the nucleic acid sequence of the genomic DNA fragment by detecting polymerization of nucleotides by a polymerase that uses the target nucleic acid molecule as a template.

### Genomic DNA Fragments

Genomic DNA fragments may be obtained from any genome, such as a human genome or a genome from any other organism. The genome may be partial or complete. The genome may be derived from any biological source, such as a patient sample or pooled patient sample; cell lines or cell cultures; biopsy material; normal tissue samples or samples from tumors or other diseased tissue; and other biological sources that would be appreciated by one skilled in the art. In one embodiment, genomic DNA is sheared, e.g., by sonication or hydrodynamic force, into fragments, generally of about 200-600 base pairs. In other embodiments, genomic DNA is fragmented by enzymatic digestion.

### Immoblization of Genomic DNA Fragments

Genomic DNA fragments may be immobilized to a solid support by any of a variety of methods. Genomic DNA fragments may be immobilized directly or indirectly to a solid support by covalent or non-covalent linkage. Suitable chemical linkers and other linkages are known to those skilled in the art. In particular embodiments, the genomic DNA fragment is denatured, wherein it is immobilized to the solid support in single-stranded form. The genomic DNA fragment, e.g., the single-stranded genomic DNA fragment, is immobilized to the solid support by way of a linking nucleic acid, or adaptor. For example, an adaptor may be ligated to one or both ends of a genomic DNA fragment, with the adaptor being immobilized to the solid support. The adaptor may be single-stranded, e.g., an oligonucleotide. In certain embodiments, the adaptor is first ligated to the genomic DNA fragment, and then the adaptor is immobilized to the solid support, or alternatively, the adaptor is first immobilized to the solid support, and then the genomic fragment is ligated to the adaptor on the solid support. In certain further embodiments, the adaptor is biotinylated (e.g., contains one or more biotinylated nucleotides), and the solid support has streptavadin on its surface, wherein the biotin moiety binds to the streptavadin, thus immobilizing the adaptor.

The orientation of the immobilized genomic DNA fragment may be 5′→3′ from the solid support, or 3′→5′ from the solid support. In one embodiment, the immobilized genomic DNA fragment is oriented 3′→5′ from the solid support. In further embodiments, the immobilized genomic DNA fragment is single-stranded. In a further embodiment, the single-stranded genomic fragment is immobilized to the solid support by way of an adaptor, e.g., an oligonucleotide. In a particular embodiment, a genomic DNA fragment is single-stranded and ligated to an adaptor which is immobilized to the solid support, wherein the adaptor and the genomic DNA fragment are oriented 3′→5′ from the solid support.

### Sequencing

The immobilized genomic DNA fragment is sequenced on the solid support. In certain embodiments, the genomic DNA fragment is immobilized on the solid support in single-stranded form, or it is immobilized on the solid support in double-stranded form, wherein it is capable of being converted in whole or in part to a single-stranded form that remains immobilized to the solid support.

In certain embodiments, the solid support is exposed to a primer under hybridization conditions, wherein the primer and genomic DNA fragment form a specific hybridization complex. In certain other embodiments, the solid support is exposed to a primer under hybridization conditions, wherein the primer and an adaptor form a specific hybridization complex. In one such embodiment, the adaptor is an oligonucleotide to which the genomic DNA fragment is ligated, wherein the adaptor is immobilized on the solid support. In the foregoing embodiments, cytosines in the nucleic acid sequence to which the primer binds in the genomic DNA fragment or the adaptor are protected from deamination resulting from bisulfite treatment, e.g., by having a protecting group. A protecting group may be a methyl group, e.g., and the protected cytosine may be 5-methylcytosine.

In further embodiments, the solid support is exposed to a polymerization reaction mixture. In one embodiment, the polymerization reaction mixture comprises a DNA polymerase and nucleotides, wherein the DNA polymerase synthesizes a nucleic acid strand from a primer using the genomic DNA fragment as a template. In one such embodiment, the DNA polymerase synthesizes a nucleic acid strand from a primer that forms a specific hybridization complex with an adaptor, wherein the adaptor (optionally) and genomic DNA fragments are used as templates. For example, if the adaptor links the genomic DNA fragment to the solid support, wherein the adaptor and the genomic DNA fragment are oriented in the 3′→5′ direction from the solid support, then the primer may hybridize to the adaptor in the 5′→3′ direction from the solid support, thereby priming synthesis by the polymerase in the 5′→3′ direction using the adaptor as a template (optionally) and using the genomic DNA fragment as a template. In another such embodiment, the DNA polymerase synthesizes a nucleic acid strand from a primer that forms a specific hybridization complex with the genomic DNA fragment, wherein the genomic DNA fragment is used as a template.

Suitable DNA polymerases include but are not limited to bacterial DNA polymerases (e.g., E. coli DNA pol I, II, III, IV, and V, and the Klenow fragment of DNA pol I); viral DNA polymerases (e.g., T4 and T7 DNA polymerases); archaeal DNA polymerases (e.g., Thermus aquaticus (Taq) DNA polymerase, Pyrococcus furiosus (Pfu) DNA polymerase, “Deep Vent” DNA polymerase (New England BioLabs)); eukaryotic DNA polymerases; and engineered or modified variants thereof.

In certain embodiments, the nucleotides and/or the polymerase in a polymerization reaction mixture are labeled. In one embodiment, one, two, three, or four types of nucleotides are differentially labeled. In one such embodiment, four different types of nucleotides are labeled with four different labels. For example, in the case of dNTPs, adenine (or a functionally equivalent analog), guanine (or a functionally equivalent analog), cytosine (or a functionally equivalent analog), and thymine (or a functionally equivalent analog) are each labeled with a different label, e.g., a different fluorophore. Suitable labels include but are not limited to luminescent, photoluminescent, electroluminescent, bioluminescent, chemiluminescent, fluorescent, and/or phosphorescent labels. Fluorescent labels include but are not limited to xanthine dye, fluorescein, cyanine, rhodamine, coumarin, acridine, Texas Red dye, BODIPY, ALEXA, GFP, and modifications thereof. A label may be directly attached to a nucleotide or may be attached via a suitable linker. A label may be attached to a nucleotide at any position that does not significantly interfere with the ability of a polymerase to incorporate the nucleotide into a growing nucleic acid strand. In one embodiment, the label is attached to a phosphate of the nucleotide, e.g., the terminal phosphate of a nucleotide, wherein the phosphate chain of the nucleotide, and therefore the label, is cleaved upon incorporation of the nucleotide into a growing nucleic acid strand. The labeled nucleotides and/or polymerases may allow for the detection of the nucleic acid strand synthesized by the polymerase.

In certain embodiments, the sequence of the genomic DNA fragment is determined by identifying the nucleotides that are incorporated into a growing nucleic acid strand by a polymerase, in the order in which they are incorporated. One embodiment comprises directly or indirectly detecting the labels of the nucleotides that are incorporated into a growing nucleic acid strand, in the order in which they are incorporated, and correlating the detected labels with the identity of the nucleotides, thereby ascertaining the sequence of the growing nucleic acid strand. Thus the sequence of the genomic DNA fragment (or the complement thereof, depending on whether the sense or antisense strand of the genomic DNA fragment is immobilized) is determined. In such embodiments, the detection of the labels, and in principle the sequencing of the genomic DNA fragment, takes place in real-time and at the “single molecule” level. It is noted above and further exemplified below that the label may be removed coincidentally with the incorporation of the nucleotide into the growing nucleic acid strand, such that the newly synthesized nucleic acid strand is not labeled.

Certain methods for detecting labeled nucleotides as they are incorporated into a growing nucleic acid strand are described in WO 2010/002939. Such methods rely on Forster Resonance Energy Transfer (FRET) between a “donor” molecule (FRET donor) and an “acceptor” molecule (FRET acceptor) when the molecules are in sufficient proximity to one another. In particular embodiments, a polymerase is labeled with a FRET donor fluorophore, and a nucleotide is labeled with a FRET acceptor fluorophore. As the polymerase incorporates the nucleotide into a growing nucleic acid strand, the FRET donor and acceptor fluorophores are brought into proximity, allowing the transfer of energy from the FRET donor fluorophore to the FRET acceptor fluorophore. The energy transfer decreases the emission intensity of the FRET donor fluorophore and increases the emission intensity of the FRET acceptor fluorophore. Detection of the emission spectrum of the FRET acceptor indicates the identity of the nucleotide being incorporated. In one embodiment, the FRET donor attached to a polymerase is a fluorescent nanoparticle, e.g., a nanocrystal, and more specifically, a quantum dot, as described in WO 2010/002939. A FRET donor may be illuminated with an excitation source such as a laser wherein the donor emission is produced. In further embodiments, a different FRET acceptor is attached to each of one or more types of nucleotides, and in particular each of three or four types of nucleotides. A FRET acceptor may be any of the fluorescent labels discussed above.

Labels may be detected using any suitable method or device including but not limited to charge couple devices and total internal reflection microscopy.

Using the above methods, multiple genomic DNA fragments may be separated and sequenced by immobilizing the genomic DNA fragments on discrete areas of a solid support, e.g., on a microarray. In this manner, genomic DNA fragments of interest may be immobilized and sequenced in a high throughput manner.

### Bisulfite Treatment and Post-Treatment Sequencing

Following sequencing of the genomic DNA fragment, the specific hybridization complex consisting of the newly synthesized DNA strand and the genomic DNA fragment is denatured (e.g., by heat or base denaturation), separating the newly synthesized DNA strand from the genomic DNA fragment. The genomic DNA fragment on the solid support is subjected to bisulfite treatment. Methods for effecting bisulfite treatment are known in the art and are described, e.g., in Herman et al. (1996) Proc. Natl. Acad. Sci. USA 93:9821-9826. Unprotected (unmethylated) cytosines in the genomic DNA fragment are thus converted to uracil. The genomic DNA fragment is then subject to a sequencing protocol as outlined above. The resulting sequence is compared with the sequence obtained prior to the bisulfite treatment to identify cytosine residues that have been converted to uracil residues in the genomic DNA fragment, as indicated, e.g., by the presence of a thymine, in place of a guanine, in the newly synthesized strand generated by the sequencing protocol. The converted residues indicate an unmethylated state in the genomic DNA fragment, whereas unconverted residues indicate a protected, i.e., methylated state in the genomic DNA fragment. In this manner, the methylation status of the genomic DNA fragment is determined.

### EXAMPLES

### [a] Direct Capture and Real Time Sequencing of DNA Molecules

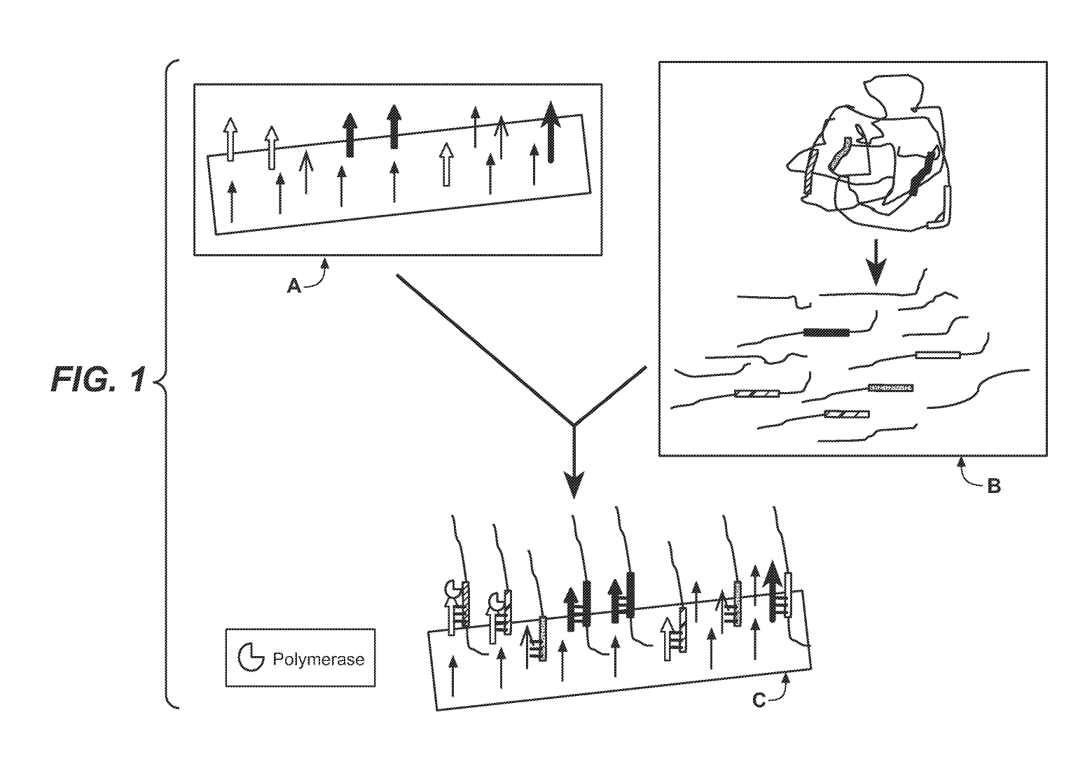

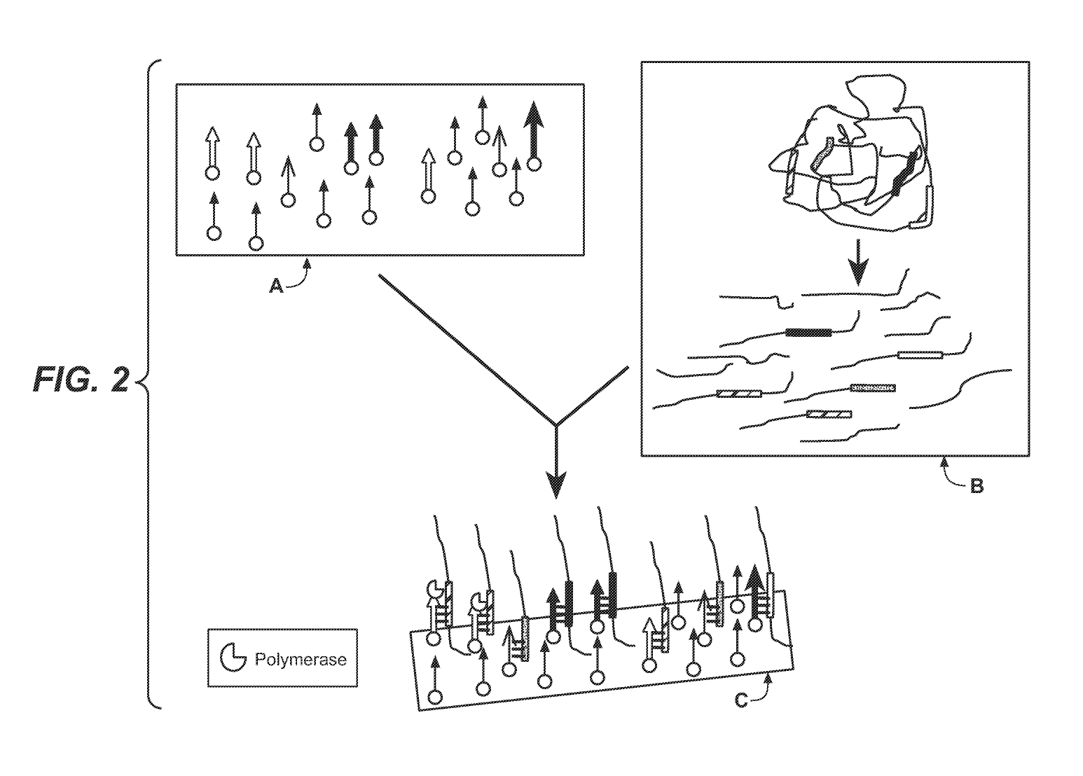

DNA containing target nucleic acid molecules of interest (e.g., genomic DNA with protein coding regions of interest) is sheared to an appropriate size, as shown in FIGS. 1B and 2B. A substrate, e.g., an array (as shown in FIG. 1A) or beads (as shown in FIG. 2A), that contains oligonucleotides complementary to the regions of interest is employed. The regions of interest may be, e.g., exons. Nucleic acids comprising the relevant regions from the sheared DNA are captured on the substrate through hybridization to the oligonucleotides. In FIG. 1, nucleic acids comprising the relevant regions from the sheared DNA are captured by hybridization to complementary oligonucleotides on an array. In FIG. 2, nucleic acids comprising the relevant regions from the sheared DNA are captured in-solution on beads. Unbound and non-specifically bound DNA is washed off. In FIG. 2C, the beads are laid onto a further substrate, e.g., an ordered or unordered array. The captured DNA is subject to direct sequencing, as shown in FIGS. 1C and 2C. This is achieved using the free 3′ ends of the oligonucleotides (which function as primers), a labeled polymerase, and labeled dNTPs that allow direct monitoring of the added bases and the polymerase during real-time synthesis of DNA at the single molecule level.

### [b] Direct Capture and Real Time Sequencing of RNA Molecules

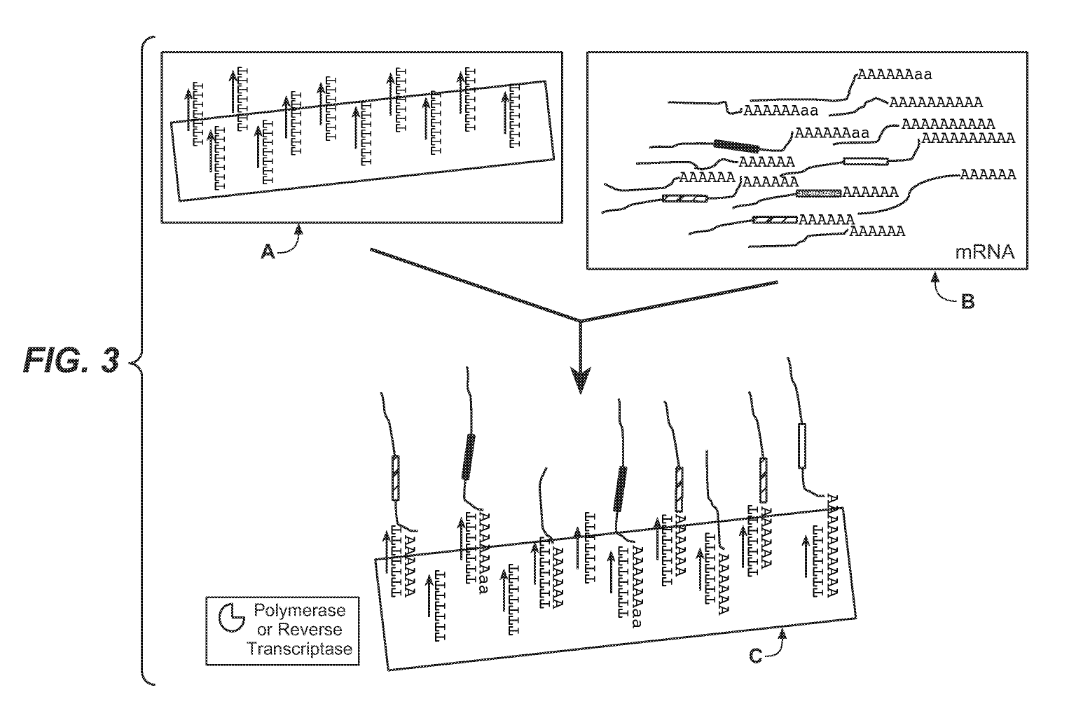

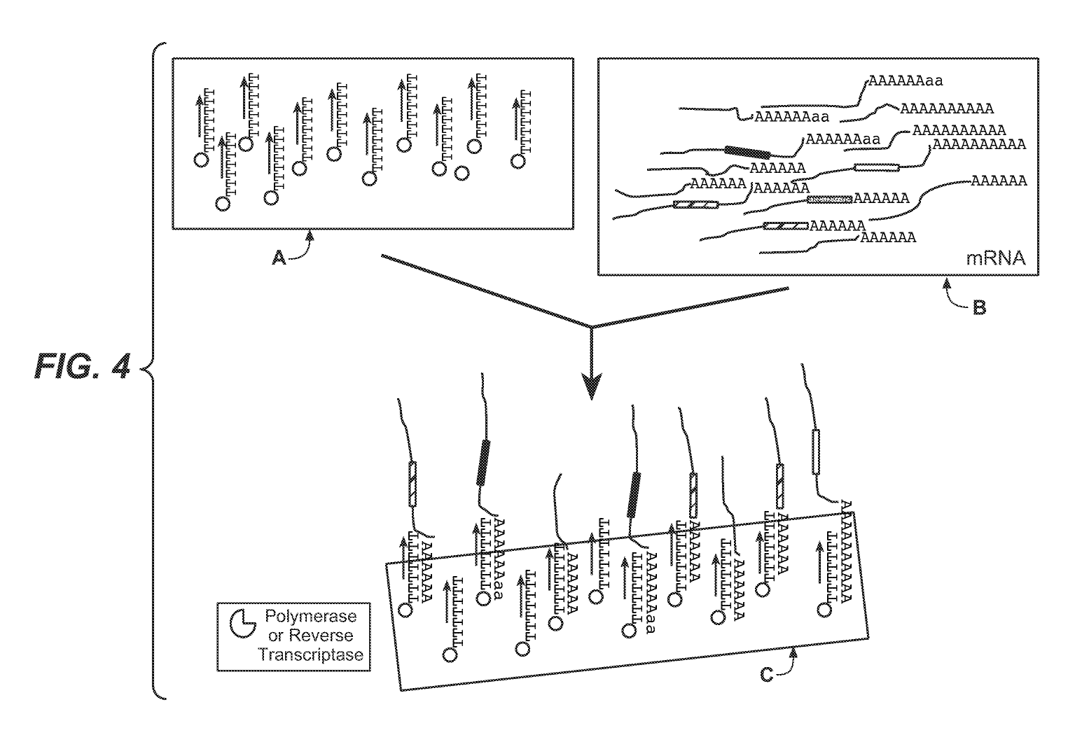

RNA containing poly-A tail may be employed, as shown in FIGS. 3B and 4B. A substrate, e.g., an array (as shown in FIG. 3A) or beads (as shown in FIG. 4A), that contains poly-dT-containing oligonucleotides is also employed. The RNA is captured on the substrate through hybridization of the poly-A tails to the poly-dT containing oligonucleotides, as shown in FIGS. 3C and 4C. Unbound and non-specifically bound RNA is washed off and the captured RNA is converted into cDNA using the free 3′ end of the poly-dT and reverse transcriptase. After conversion of RNA to cDNA, the cDNA is sequenced. In one embodiment for sequencing the cDNA, an oligonucleotide adapter is ligated to the free 3′ end of the newly synthesized cDNA, and then a primer is annealed to that adapter. The cDNA is sequenced using that primer, a labeled polymerase and labeled dNTPs that allow direct monitoring of the added bases and the polymerase during real-time synthesis of DNA at the single molecule level. Alternatively, the captured RNA can be sequenced directly using a labeled reverse transcriptase and labeled dNTPs that allow direct monitoring of the added bases and the reverse transcriptase during real-time synthesis of the cDNA at the single molecule level. (See FIGS. 3C and 4C.)

### [c] Methylation Status Determination by Recursive Sequencing of the Same Template

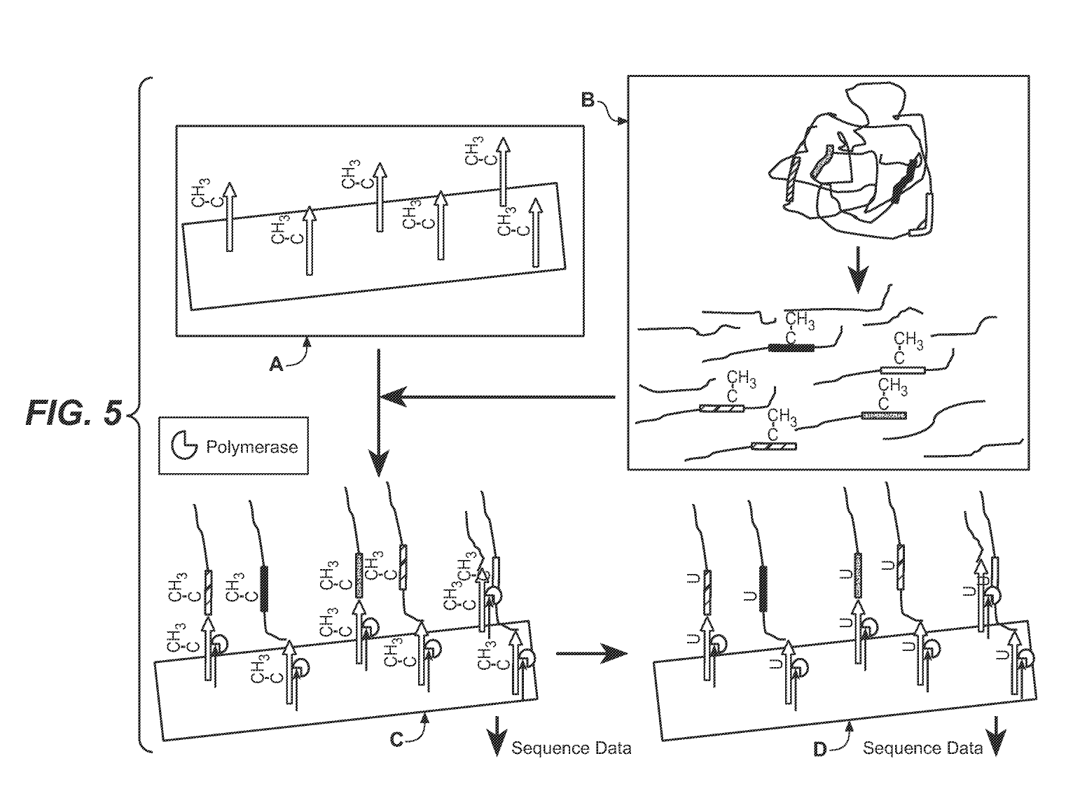

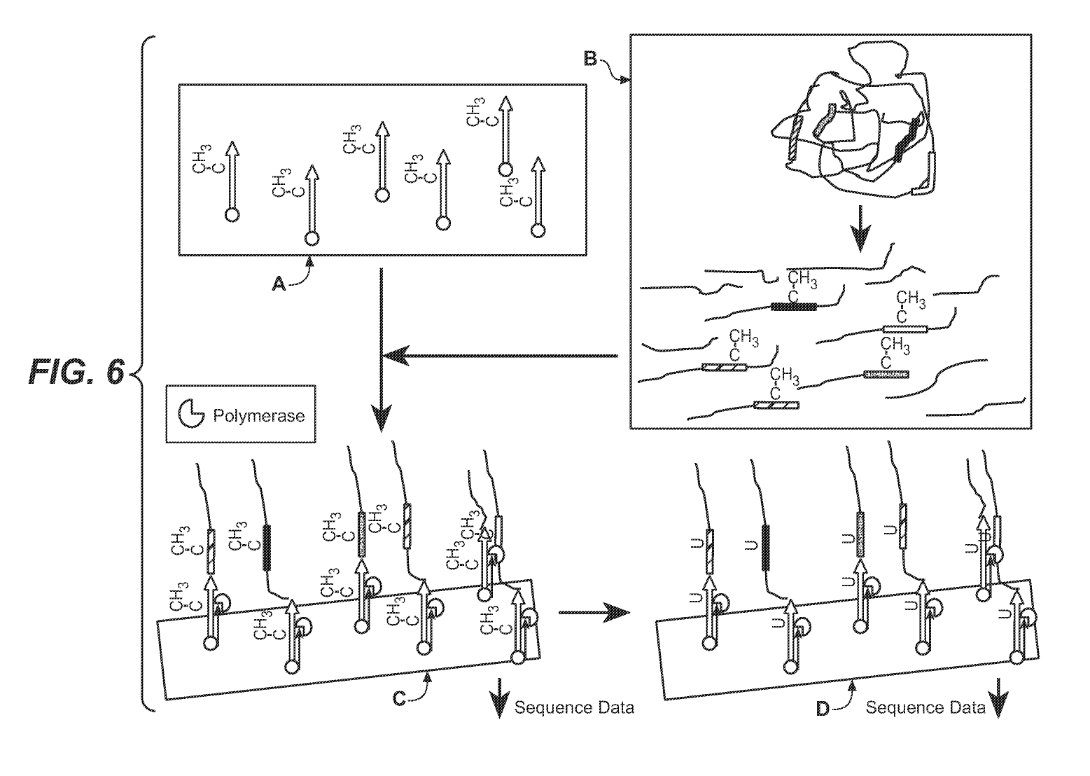

To determine the methylation status of DNA (e.g., genomic DNA), it is first sheared to an appropriate size and preferably converted to single strand, as shown in FIGS. 5B and 6B. A substrate, e.g., an array (as shown in FIG. 5A) or beads (as shown in FIG. 6A), that contains methylated oligonucleotides (i.e., methylcytosine-containing oligonucleotides) is employed. The sheared DNA is ligated to the methylated oligonucleotides, as shown in FIGS. 5C and 6C. The ligated DNA is then sequenced using a primer complementary to the methylated oligonucleotide on the array or bead, a labeled polymerase, and labeled dNTPs that will allow direct monitoring of the added bases and the polymerase during real-time synthesis of DNA at the single molecule level. After sequencing the ligated DNA, the newly synthesized strand is removed and the original DNA is treated with bisulfite to allow conversion of methylated cytosines to uracil, as shown in FIGS. 5D and 6D. The treated DNA is sequenced using a primer, labeled polymerase and labeled dNTPs that will allow direct monitoring of the added bases and the polymerase during real-time synthesis of DNA at the single molecule level. Comparison of the sequence obtained before and after treatment with bisulfite from the same molecule will allow determination of the methylation status of the DNA.

All patents and publications cited herein are incorporated by reference.

What is claimed is:

1. A method of capturing and sequencing a target nucleic acid molecule comprising:
(a) exposing a solid support to a mixture of nucleic acids comprising the target nucleic acid molecule under hybridizing conditions, wherein the target nucleic acid molecule forms a specific hybridization complex with a primer immobilized on the solid support in a priming-competent configuration;
(b) separating unbound and non-specifically bound nucleic acids from the solid support;
(c) exposing the solid support to a polymerase and nucleotides under polymerization conditions; and
(d) determining a nucleic acid sequence of the target nucleic acid molecule by detecting nucleic acid polymerization from the immobilized primer by the polymerase using the target nucleic acid molecule as a template.

2. The method of claim 1, wherein the target nucleic acid molecule is from a region of genomic DNA.

3. The method of claim 2, wherein the target nucleic acid comprises all or part of an exon.

4. The method of claim 1, wherein the target nucleic acid molecule is RNA, the polymerase is a reverse transcriptase, and the primer comprises a 3′ poly-T sequence.

5. The method of claim 1, wherein the target nucleic acid molecule is DNA, and the polymerase is a DNA polymerase.

6. The method of claim 1, wherein the nucleotides are labeled at their terminal phosphates.

7. The method of claim 6, wherein the polymerase is labeled with a FRET donor, and the nucleotides are labeled with a FRET acceptor.

8. The method of claim 7, wherein the FRET donor is a fluorescent nanoparticle.

9. A method of determining the methylation status of a genomic DNA fragment, the method comprising:
(a) immobilizing a genomic DNA fragment on a solid support;
(b) determining a nucleic acid sequence of the immobilized genomic DNA fragment on the solid support by detecting nucleic acid polymerization by a polymerase using the immobilized genomic DNA fragment as a template;
(c) subjecting the immobilized genomic DNA fragment to bisulfite treatment;
(d) determining a nucleic acid sequence of the immobilized, bisulfite-treated genomic DNA fragment on the solid support by detecting nucleic acid polymerization by a polymerase using the immobilized, bisulfite-treated genomic DNA fragment as a template;
(e) comparing the nucleic acid sequence determined in (b) with the sequence determined in (d), wherein conversion of a cytosine residue in the genomic DNA fragment indicates that the residue was unmethylated in the genomic DNA fragment prior to the bisulfite treatment, and wherein absence of conversion of a cytosine residue in the genomic DNA fragment indicates that the residue was methylated in the genomic DNA fragment prior to the bisulfite treatment.

10. The method of claim 9, wherein the genomic DNA fragment is immobilized to the solid support by an adaptor.

11. The method of claim 10, wherein the adaptor contains a primer binding site, and wherein cytosines in the primer binding site are protected.

12. The method of claim 11, wherein the polymerase of (b) and/or (d) polymerizes a nucleic acid strand from a primer annealed to the primer binding site.

13. The method of claim 9, wherein nucleic acid polymerization in (b) and/or (d) is detected by detecting the incorporation of labeled nucleotides.

14. The method of claim 13, wherein the labeled nucleotides are labeled at their terminal phosphates.

15. The method of claim 14, wherein the polymerase of (b) and/or (d) is labeled with a FRET donor, and the nucleotides are labeled with a FRET acceptor.

16. The method of claim 15, wherein the FRET donor is a fluorescent nanoparticle.
